(19) United States
(12) Patent Application Publication
(10) Pub. No.: US 2026/0051371 A1
(43) Pub. Date: Feb. 19, 2026

(54) TRAINING MACHINE LEARNING MODELS TO PREDICT PROPERTIES OF MOLECULES

(71) Applicant: International Business Machines Corporation, Armonk, NY (US)

(72) Inventors: Indrapriyadarsini Sendilkkumaar, Kanagawa (JP); Akihiro Kishimoto, Tokyo (JP); Seiji Takeda, Tokyo (JP)

(21) Appl. No.: 18/806,191

(22) Filed: Aug. 15, 2024

(51) Int. Cl. G16C 20/70; G16C 20/30; G16C 20/50

(52) U.S. Cl. CPC G16C 20/70; G16C 20/30; G16C 20/50

(57) ABSTRACT

Systems, methods, and computer program products for training a machine learning model are described herein. A method may comprise reading a representation characterizing a structure of a molecule, providing the first representation as input to a representation generator, reading a plurality of alternative representations generated by the representation generator, providing the plurality of alternative representations as input to an autoencoder, reading a plurality of latent representations generated by the autoencoder responsive to receipt of the representation as input, each of the plurality of patent representations individually corresponding to one of the plurality of alternative representations, aggregating at least some of the plurality of latent representations to generate an aggregate latent representation, and providing the aggregate latent representation as input for a prediction machine learning model configured to predict values for properties of molecules based on input representations.

### BACKGROUND

Embodiments of the present disclosure relate to predicting properties of molecules, and more specifically, to training a machine learning to predict properties of molecules.

### SUMMARY

According to embodiments of the present disclosure, computer-implemented methods, computer program products, and computer systems are disclosed. A computer-implemented method for training a machine learning model to predict properties of molecules is disclosed. The method may include reading a first representation of a molecule. The first representation may characterize a structure of the molecule. The computer-implemented method may include providing the first representation as input to a representation generator. The computer-implemented method may include reading a plurality of alternative representations of the molecule generated by the representation generator based on the first representation. The computer-implemented method may include providing the plurality of alternative representations as input to an autoencoder. The computer-implemented method may include reading a plurality of latent representations generated by the autoencoder. The plurality of latent representations may be generated responsive to receipt of the representation as input. Each of the plurality of patent representations may correspond to one of the plurality of alternative representations. The computer-implemented method may include aggregating at least some of the plurality of latent representations to generate an aggregate latent representation. The computer-implemented method may include providing the aggregate latent representation as input for a prediction machine learning model. The prediction machine learning model may be configured to predict values for properties of molecules based on input representations.

### BRIEF DESCRIPTION OF THE DRAWINGS

FIG. 1 is a flowchart illustrating a method for training a machine learning model to predict properties of molecules, in accordance with one or more embodiments of this disclosure.

FIG. 2 is a block diagram depicting an exemplary method for training a machine learning model to predict properties of molecules, in accordance with one or more embodiments of this disclosure.

FIG. 3 is a schematic diagram of a computing node, in accordance with one or more embodiments of this disclosure.

### DETAILED DESCRIPTION

Predicting properties of molecules using machine learning models enables faster material discovery. Additionally, such predictions minimize the need for physical testing of new molecules for determining properties of the molecules. However, the availability of datasets of molecules for training machine learning models to learn to predict properties of molecules is limited. Training machine learning models to effectively generate latent representations of molecules requires diverse sets of molecules and representations for the molecules. As such, current methods for training machine learning models to predict properties of molecules require using data augmentation (e.g., SMILES enumeration) to generate a training dataset. Such data augmentation does not guarantee quality or expressiveness of the latent representations learned by the model. As such, a method that guarantees and/or improves the quality of the latent representations learned by autoencoders using current datasets is necessary. One such method, as described herein, is aggregating latent representations generated for a plurality of representations of the same molecule to generate an aggregate latent representation of the molecule. The aggregate latent representation may be an enriched feature vector characterizing the molecule. In particular, the use of multiple representations for generating the aggregate latent representation enables the aggregate latent representation to encode information characterizing multiple views of the molecule.

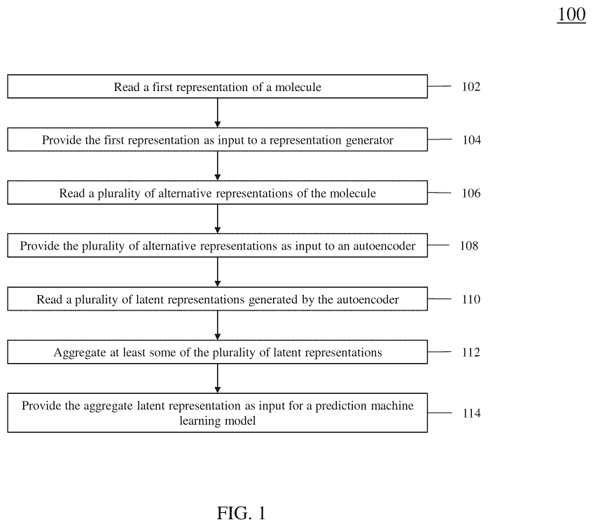

FIG. 1 is a flowchart illustrating an exemplary method 100 for optically processing data. The operations of method 100 presented below are intended to be illustrative. In some implementations, method 100 may be accomplished with one or more additional operations not described and/or without one or more of the operations discussed. Additionally, the order in which the operations of method 100 are illustrated in FIG. 1 and described below is not intended to be limiting.

In some implementations, method 100 may be implemented in one or more processing devices (e.g., a digital processor, an analog processor, a digital circuit designed to process information, a state machine, and/or other mechanisms for electronically processing information). The one or more processing devices may include one or more devices configured through hardware, firmware, and/or software to be specifically designed for execution of one or more of the operations of method 100.

Operation 102 includes reading a first representation of a molecule. The first representation may characterize a structure of the molecule. By way of non-limiting example, the first representation may represent graph structure of the molecule. For example, the first representation is in the form of a simplified molecular-input line-entry system (SMILES) strings, a self-referencing embedded strings (SELFIES), and/or another form for representing molecular structure. By way of non-limiting example, the first representation is the canonical SMILES representation of the molecule.

Operation 104 includes providing the first representation as input to a representation generator. The representation generator may be configured to generate a plurality of alternative representations of the molecule. The plurality of alternative representations may be generated based on the first representation. The alternative representations may be strings of characters. For example, the alternative representations are in the form of SMILES strings, SELFIES, and/or other forms for representing molecular structure. Individual ones of the alternative representations may be in the same form as the first representation or in different forms than the first representation.

The representation generator may randomly shuffle the characters of the first representation to generate strings of characters that are representative of the structure of the molecule. By way of non-limiting example, the characters of the first representation may be repeatedly shuffled until a desired number of valid alternative representations of the molecule are generated. In some implementations, the representation generator may be configured to generate the plurality of alternative representations using RDKit. By way of non-limiting example, the representation generator is configured to generate non-canonical representations of the molecule based on the first representation using RDKit. Operation 106 includes reading the plurality of alternative representations of the molecule generated by the representation generator based on the first representation.

Operation 108 includes providing the plurality of alternative representations as input to an autoencoder. In various embodiments, a plurality of vectors of features that characterize the plurality of alternative representations may be provided to the autoencoder. For example, the vectors of features may be tokenized forms of the alternative representations. As used herein, reference to providing the plurality of alternative representations as input to the autoencoder may refer to providing each of the plurality of vectors of features as input to the autoencoder. Based on the features of an individual vector, the autoencoder may generate one or more outputs. In some implementations, the output(s) of the autoencoder may comprise a vector of features. By way of non-limiting example, the autoencoder and/or individual portions of the autoencoder may generate a vector of features characterizing a string of characters. The generated vector of features may be converted to the string of characters.

The autoencoder may comprise an encoder and a decoder. The autoencoder may use a convolutional neural network (CNN) architecture, a recurrent neural network (RNN) architecture (e.g. a long short-term memory architecture), a transformer architecture, a feed-forward neural network, and/or another neural network architecture. In some implementations, the autoencoder may be pretrained. In some implementations, pretraining the autoencoder comprises providing representations of molecules to the autoencoder as input. The autoencoder may be pretrained via unsupervised learning to reconstruct the input representations of the molecules. For example, SMILES strings and SELFIES are provided to the autoencoder during pretraining. The autoencoder may be pretrained to generate the same SMILES strings and SELFIES as provided as input.

Operation 110 includes reading a plurality of latent representations generated by the autoencoder. The plurality of latent representations may be generated responsive to receipt of the representation as input. In some implementations, the encoder of the autoencoder generates the plurality of latent representations. Each of the plurality of latent representations may individually correspond to one of the plurality of alternative representations. The latent representations may be tensors of any dimension. For example, the latent representations are vectors. The latent representations may characterize one or more features of the molecule.

Operation 112 includes aggregating at least some of the plurality of latent representations to generate an aggregate latent representation. The aggregate latent representation may be a feature vector characterizing the molecule. The aggregate latent representation may encode more information characterizing the molecule than an individual latent representation. Aggregating the plurality of latent representations may comprise concatenating the at least some of the plurality of latent representations. In some implementations, aggregating the at least some of the plurality of latent representations comprises selecting from the plurality of latent representations to determine the at least some of the latent representations. By way of non-limiting example, the at least some of the plurality of latent representations is a subset of the plurality of latent representations. Operation 112 aggregating the at least some of the plurality of latent representations may comprise selecting from the plurality of latent representations to determine the subset of the plurality of latent representations. Only some of the latent representations may be selected to limit the size of an aggregation of latent representations. In some implementations, an individual latent representation is included in the subset based on features encoded by the latent representation, a quality of features encoded by the latent representation, and/or other characteristics of the latent representation. In some implementations, determining the at least some of the latent representations comprises a greedy search of the latent representations. By way of non-limiting example, the at least some of the plurality of latent representations comprises each latent representation of the plurality of latent representations.

Operation 114 includes providing the aggregate latent representation as input for a prediction machine learning model. The prediction machine learning model may be configured to predict values for properties of molecules based on input representations. In some implementations, the properties comprise one or more of chemical properties, physical properties, structural properties, and/or other types of properties. In some implementations, the values comprise levels of properties of molecules. By way of non-limiting example, prediction machine learning model is configured to generate a level of toxicity of the molecule. In some implementations, the values comprise characterizations of whether molecules have particular properties. By way of non-limiting example, prediction machine learning model is configured to determine whether the molecule is toxic. The prediction machine learning model may generate a value of a property responsive to providing the aggregate latent representation as input.

In various embodiments, the prediction machine learning model may be and/or may include a dynamic programming algorithm and/or model, such as a dynamic linear programming algorithm/model or a dynamic nonlinear programming algorithm/model. In various embodiments, the one or more machine learning models, described herein, may be a trained classifier. In various embodiments, the trained classifier may be a random decision forest. However, it will be appreciated that a variety of other classifiers are suitable for use according to the present disclosure, including linear classifiers, support vector machines (SVM), or artificial neural network models, such as generative adversarial networks (GANs) and/or recurrent neural networks (RNNs).

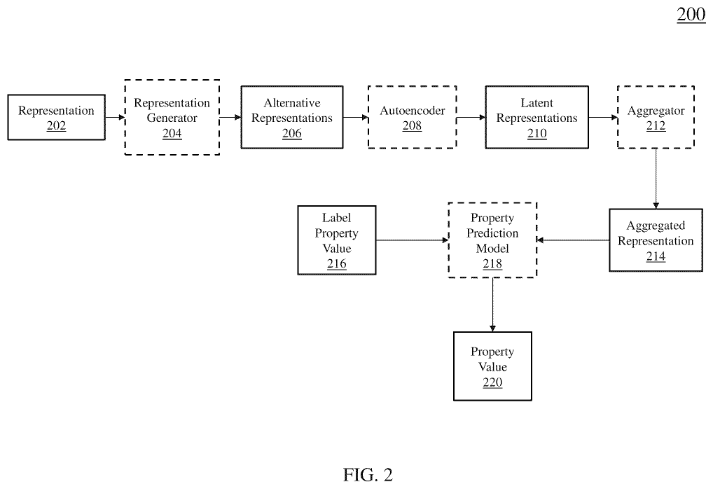

FIG. 2 is a block diagram demonstrating a process 200 for training a machine learning model to predict properties of molecules, according to an exemplary embodiment of the present disclosure. Representation 202 may be provided to representation generator 204 as input. Representation generator 204 may generate and/or output alternative representations 206 based on representation 202. Alternative representations 206 may be provided to autoencoder 208 as input. Autoencoder 208 may generate and/or output latent representations 210 based on alternative representations 206. Latent representations 210 may be provided as input to aggregator 212. Aggregator 212 may aggregate at least some latent representations 210 to generate aggregated representation 214. Aggregator 212 may be a computer component or program configured to aggregate latent representations 210. Aggregator 212 may concatenate the at least some alternative representations 206. Aggregated representation 214 may be provided as input to property prediction model 218. Property prediction model 218 may generate and/or output property value 220 based on aggregated representation 214.

Property prediction model 218 may be a machine learning model that is the same as or similar to the machine learning model described with reference to FIG. 1. In such implementations, label property value 216 is also provided to property prediction model 218 as input. In some implementations, property prediction model 218 is trained via supervised learning. In such an implementation, label property value 216 is a label for an input of aggregated representation 214. By way of non-limiting example, label property value 216 may be a measured value of a property of a molecule.

Training property prediction model 218 may comprise modifying and testing performance of property prediction model 218. Process 200 may be repeated for a training set of representations 202 and label property values 216 until a desired level of accuracy is reached. Such a process may be repeated for a set of aggregated representations 214 and corresponding label property values 216 until a desired level of accuracy is reached. Modifying property prediction model 218 may comprise adjusting one or more weights of property prediction model 218.

Testing performance of property prediction model 218 may comprise determining a current level of accuracy of property prediction model 218. Determining a current level of accuracy may comprise using a validation plurality of representations 202, a validation plurality of aggregated representations 214, and/or a validation plurality of label property values 216. Determining the current level of accuracy may comprise comparing output property values 220 to individual ones of the validation plurality of label property values 216. Such output property values 220 may have been generated by property prediction model 218 responsive to receipt of the validation plurality of label property values as input. By way of non-limiting example, a loss function is used to compare output property values 220 and the individual ones of the validation plurality of label property values 216.

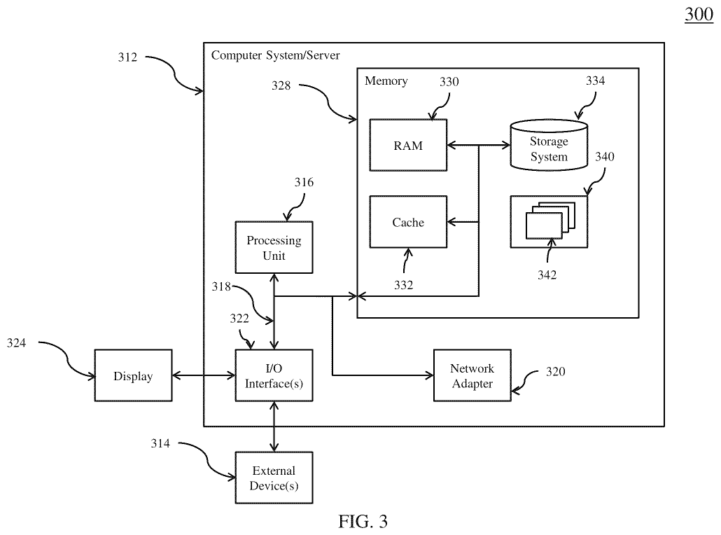

Referring now to FIG. 3, a schematic of an example of a computing node is shown. Computing node 310 is only one example of a suitable computing node and is not intended to suggest any limitation as to the scope of use or functionality of embodiments described herein. Regardless, computing node 310 is capable of being implemented and/or performing any of the functionality set forth hereinabove.

In computing node 310 there is a computer system/server 312, which is operational with numerous other general purpose or special purpose computing system environments or configurations. Examples of well-known computing systems, environments, and/or configurations that may be suitable for use with computer system/server 312 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like. Computer system/server 312 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 312 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 3, computer system/server 312 in computing node 310 is shown in the form of a general-purpose computing device. The components of computer system/server 312 may include, but are not limited to, one or more processors or processing units 316, a system memory 328, and a bus 318 that couples various system components including system memory 328 to processor 316.

Bus 318 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 312 typically includes a variety of computer system readable media. Such media may be any available media that is accessible by computer system/server 312, and it includes both volatile and non-volatile media, removable and non-removable media.

System memory 328 can include computer system readable media in the form of volatile memory, such as random access memory (RAM) 330 and/or cache memory 332. Computer system/server 312 may further include other removable/non-removable, volatile/non-volatile computer system storage media. By way of example only, storage system 334 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 318 by one or more data media interfaces. As will be further depicted and described below, memory 328 may include at least one program product having a set (e.g., at least one) of program modules that are configured to carry out the functions of embodiments of the disclosure.

Program/utility 340, having a set (at least one) of program modules 342, may be stored in memory 328 by way of example, and not limitation, as well as an operating system, one or more application programs, other program modules, and program data. Each of the operating system, one or more application programs, other program modules, and program data or some combination thereof, may include an implementation of a networking environment. Program modules 342 generally carry out the functions and/or methodologies of embodiments as described herein.

Computer system/server 312 may also communicate with one or more external devices 314 such as a keyboard, a pointing device, a display 324, etc.; one or more devices that enable a user to interact with computer system/server 312; and/or any devices (e.g., network card, modem, etc.) that enable computer system/server 312 to communicate with one or more other computing devices. Such communication can occur via Input/Output (I/O) interfaces 322. Still yet, computer system/server 312 can communicate with one or more networks such as a local area network (LAN), a general wide area network (WAN), and/or a public network (e.g., the Internet) via network adapter 320. As depicted, network adapter 320 communicates with the other components of computer system/server 312 via bus 318. It should be understood that although not shown, other hardware and/or software components could be used in conjunction with computer system/server 312. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer-readable storage medium (or media) having computer-readable program instructions thereon for causing a processor to carry out aspects of the present disclosure.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference has been made in detail herein to the exemplary embodiments of the present disclosure, examples of which are illustrated in the accompanying drawings. The systems, devices, and methods disclosed herein are described in detail by way of examples, and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

For any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for training a machine learning model to predict properties of molecules:
reading a first representation of a molecule, wherein the first representation characterizes a structure of the molecule;
providing the first representation as input to a representation generator;
reading a plurality of alternative representations of the molecule generated by the representation generator based on the first representation;
providing the plurality of alternative representations as input to an autoencoder;
reading a plurality of latent representations generated by the autoencoder responsive to receipt of the representation as input, each of the plurality of patent representations individually corresponding to one of the plurality of alternative representations;
aggregating at least some of the plurality of latent representations to generate an aggregate latent representation; and
providing the aggregate latent representation as input for a prediction machine learning model, wherein the prediction machine learning model is configured to predict values for properties of molecules based on input representations.

2. The computer-implemented method of claim 1, wherein aggregating the plurality of latent representations comprises concatenating the at least some of the plurality of latent representations.

3. The computer-implemented method of claim 1, further comprising:
selecting from the plurality of latent representations to determine the at least some of the latent representations.

4. The computer-implemented method of claim 3, wherein selecting from the plurality of latent representations comprises a greedy search.

5. The computer-implemented method of claim 1, further comprising:
generating, by the prediction machine learning model, a value of a property responsive to providing the aggregate latent representation as input.

6. The computer-implemented method of claim 1, wherein the prediction machine learning model is an untrained machine learning model, wherein the computer-implemented method further comprises:
providing a label property value to the prediction machine learning model, wherein the label property value characterizes a level of a property of the molecule; and
training the prediction machine learning model based in part on the aggregate latent representation and the label property value.

7. The computer-implemented method of claim 1, wherein the alternative representations are strings of characters.

8. The computer-implemented method of claim 1, wherein the first representation is in the form of a simplified molecular-input line-entry system (SMILES) string or a self-referencing embedded string (SELFIES).

9. The computer-implemented method of claim 8, wherein the first representation is the canonical simplified molecular-input line-entry system representation of the molecule.

10. The computer-implemented method of claim 1, wherein the latent representations are vectors characterizing one or more features of the molecule.

11. The computer-implemented method of claim 1, wherein generating the plurality of alternative representations comprises:
randomly shuffling characters of the first representation to generate strings of characters that are representative of the structure of the molecule.

12. The computer-implemented method of claim 1, wherein generating the plurality of alternative representations comprises:
generating the plurality of alternative representations based on the first representation using RDKit.

13. A computer program product for training a machine learning model to predict properties of molecules, the computer program product comprising:
a set of one or more computer-readable storage media; and
program instructions, collectively stored in the set of one or more storage media for causing a processor set to perform the following computer operations:
read a first representation of a molecule, wherein the first representation characterizes a structure of the molecule,
provide the first representation as input to a representation generator,
read a plurality of alternative representations of the molecule generated by the representation generator based on the first representation,
provide the plurality of alternative representations as input to an autoencoder,
read a plurality of latent representations generated by the autoencoder responsive to receipt of the representation as input, each of the plurality of patent representations individually corresponding to one of the plurality of alternative representations,
aggregate at least some of the plurality of latent representations to generate an aggregate latent representation, and
provide the aggregate latent representation as input for a prediction machine learning model, wherein the prediction machine learning model is configured to predict values for properties of molecules based on input representations.

14. The computer program product of claim 13, wherein aggregating the plurality of latent representations comprises concatenating the at least some of the plurality of latent representations.

15. The computer program product of claim 13, wherein the computer operations further comprise:
select from the plurality of latent representations to determine the at least some of the latent representations.

16. The computer program product of claim 13, wherein the prediction machine learning model is an untrained machine learning model, wherein the computer operations further comprise:
provide a label property value to the prediction machine learning model, wherein the label property value characterizes a level of a property of the molecule, and
train the prediction machine learning model based in part on the aggregate latent representation and the label property value.

17. A computer system for obfuscating search queries, the computer system comprising:
a processor set;
a set of one or more computer-readable storage media; and
program instructions, collectively stored in the set of one or more storage media for causing the processor set to perform the following computer operations:
read a first representation of a molecule, wherein the first representation characterizes a structure of the molecule,
provide the first representation as input to a representation generator,
read a plurality of alternative representations of the molecule generated by the representation generator based on the first representation,
provide the plurality of alternative representations as input to an autoencoder,
read a plurality of latent representations generated by the autoencoder responsive to receipt of the representation as input, each of the plurality of patent representations individually corresponding to one of the plurality of alternative representations,
aggregate at least some of the plurality of latent representations to generate an aggregate latent representation, and
provide the aggregate latent representation as input for a prediction machine learning model, wherein the prediction machine learning model is configured to predict values for properties of molecules based on input representations.

18. The computer system of claim 17, wherein aggregating the plurality of latent representations comprises concatenating the at least some of the plurality of latent representations.

19. The computer system of claim 17, wherein the computer operations further comprise:
select from the plurality of latent representations to determine the at least some of the latent representations.

20. The computer system of claim 17, wherein the prediction machine learning model is an untrained machine learning model, wherein the computer operations further comprise:
provide a label property value to the prediction machine learning model, wherein the label property value characterizes a level of a property of the molecule, and
train the prediction machine learning model based in part on the aggregate latent representation and the label property value.
